(19) United States
(12) Patent Application Publication
(10) Pub. No.: US 2016/0262822 A1
(43) Pub. Date: Sep. 15, 2016

(54) MEDICAL DEVICE

(71) Applicant: TERUMO KABUSHIKI KAISHA, Tokyo (JP)

(72) Inventor: Yukitoshi KATOU, Hadano-city (JP)

(73) Assignee: TERUMO KABUSHIKI KAISHA, Tokyo (JP)

(21) Appl. No.: 15/065,980

(22) Filed: Mar. 10, 2016

(30) Foreign Application Priority Data
Mar. 13, 2015 (JP) 2015-050449

(51) Int. Cl. A61B 18/08

(52) U.S. Cl. CPC A61B 18/082; A61B 2018/00577

(57) ABSTRACT

A medical device includes: an elongate outer sheath; an inside shaft portion extending through an inside of the outer sheath to protrude distally beyond the outer sheath; a heating portion located between a distal portion of the outer sheath and a distal portion of the inside shaft portion, the heating portion provided with a plurality of linear material portions extending in an axial direction of the inside shaft portion; and leads electrically connected to the heating portion for passing a current to the heating portion. The linear material portion includes a conduction portion and a coating portion covering the linear material portion. The heating portion can be expanded by moving the inside shaft portion in the axial direction relative to the outer sheath. The temperature of the heating portion rises owing to electric resistance when a current flows through the heating portion.

### CROSS-REFERENCES TO RELATED APPLICATIONS

This application claims priority to Japanese Patent Application No. 2015-050449 filed on Mar. 13, 2015, the entire content of which is incorporated herein by reference.

### TECHNICAL FIELD

The present disclosure generally relates to a medical device for heating a body lumen.

### BACKGROUND DISCUSSION

There is a known method of heating a body lumen, such as a blood vessel, that involves inserting a device in the body lumen. One example of such a method is ablation treatment in which a vein is cauterized from inside the vein. Veins are provided with venous valves for preventing backflow of blood. Veins in the lower limb are contracted by muscles of the lower limb, and thus function also as a pump for returning blood to the heart against the effect of gravity. When a venous valve becomes unable to operate normally, backflow of blood occurs in the vein and the vein becomes enlarged, which may cause a varicose vein. Varicose veins often occur in the great saphenous vein and the small saphenous vein, which are superficial veins in the lower limb that are subject to high pressure in a standing position. If a venous valve in a superficial vein stops operating normally, the blood which normally flows from the superficial vein into a deep vein would flow in the reverse direction from the deep vein into the superficial vein. The reverse blood flow can cause the superficial vein to become enlarged, and cause a tortuous varicose vein. In view of this, a therapy is conducted in which a device provided with a heating element is inserted into the vein, and the vein is cauterized by heat, so as to occlude (i.e., block) the varicose vein.

For example, U.S. Patent Application Publication No. 2009/0281535 describes a medical device in which a pair of electrodes are disposed in spiral form at an outer peripheral surface of a pipe body. The medical device can be inserted into a vein, and can perform RF (radio frequency) ablation by impressing an RF current to the paired electrodes. In the use of this device, after the electrodes are inserted into a blood vessel, the blood vessel is compressed from outside the body to bring the electrodes into contact with the inner wall of the blood vessel.

### SUMMARY

In RF ablation treatment, the electrodes may be heated to about 120° C. TLA (tumescent local anesthesia) is applied to the surroundings of the treatment site for mitigating the pain (i.e., felt by the patient) due to the heat. However, this treatment takes a lot of time because the TLA fluid is injected into the surroundings of the treatment site a number of times (i.e., multiple injections of the TLA fluid are necessary).

The medical device disclosed here permits lowering of a heating temperature for a body lumen. This medical device can enhance safety, and the body lumen can be heated through a simple operation.

In one aspect, a medical device includes: an elongate outer sheath; an inside shaft portion extending through an inside of the outer sheath to protrude distally beyond the outer sheath; a heating portion located between a distal portion of the outer sheath and a distal portion of the inside shaft portion, the heating portion provided with a plurality of linear material portions extending in an axial direction of the inside shaft portion, the heating portion formed in a tubular shape; and leads electrically connected to the heating portion for passing a current to the heating portion. The linear material portion includes a conduction portion configured to conduct a current, and a coating portion covering an outer peripheral surface of the linear material portion. The coating portion is an insulating material. In addition, the heating portion is expandable radially outward by moving the inside shaft portion in the axial direction relative to the outer sheath, and the temperature of the heating portion increases when a current flows through the heating portion.

In another aspect, the medical device includes an outer sheath and an inner shaft portion extending through the inner portion of the outer sheath in an axial direction to protrude distally beyond the outer sheath. The inner shaft portion is slidably movable relative to the outer sheath. The inner shaft portion includes a distal portion located distal to the distal end of the outer sheath in the axial direction. The medical device also includes a heating portion connected to the distal end of the outer sheath and the distal portion of the inner shaft portion, the heating portion including a braided mesh. The braided mesh is formed by a plurality of linear material portions extending in the axial direction of the inner shaft portion, each of the linear material portions has a spiral shape and includes a conduction portion configured to conduct current to increase the temperature of the heating portion and a coating portion covering an outer peripheral surface of the conduction portion. The coating portion is an insulating material. The braided mesh of the heating portion is expandable radially outward when the inner shaft portion moves in the axial direction relative to the outer sheath, and the temperature of the heating portion increases when the conduction portion conducts current to increase the temperature of the heating portion.

In the medical device configured as described above, heating can be achieved by passing a current to the heating portion which can deform while bending to the radially outer side. Therefore, the heating portion can generate heat when the heating portion is put in contact with the body lumen wall through deformation, so that the body lumen can be heated directly. Accordingly, the body lumen can be heated effectively, which ensures that the heating temperature can be lowered, and safety can be thereby enhanced. In addition, it is unnecessary to compress the blood vessel from outside the body, and the body lumen can thus be heated by a simple operation.

In a further aspect, there is provided a method including: inserting a medical device into a living body, the medical device comprising a heating portion, the heating portion being expandable; moving the medical device to a body lumen within the living body, the body lumen possessing an inner wall and an inner diameter; expanding the heating portion of the medical device to contact the inner wall of the body lumen; heating the inner wall of the body lumen with the heating portion of the medical device while the heating portion is contacting the inner wall of the body lumen; and contracting the inner wall of the body lumen through the heating of the inner wall of the body lumen with the heating portion so that the inner diameter of the body lumen decreases.

### BRIEF DESCRIPTION OF THE DRAWINGS

FIG. 1 is a plan view showing a medical device according to a first embodiment.

FIG. 2 is a sectional view showing a distal portion of the medical device according to the first embodiment.

FIG. 3 is a plan view showing a condition where a heating portion of the medical device according to the first embodiment is expanded.

FIGS. 4A and 4B are schematic sectional views illustrating a first treatment method, wherein FIG. 4A shows a condition where a guide wire is inserted in a varicose vein, and FIG. 4B shows a state before expansion of the heating portion.

FIGS. 5A and 5B are schematic sectional views illustrating the first treatment method, wherein FIG. 5A shows a condition where the heating portion is expanded in the varicose vein, and FIG. 5B shows a condition where the varicose vein is heated by the heating portion.

FIGS. 6A and 6B are schematic sectional views illustrating the first treatment method, wherein FIG. 6A shows a state when the heating portion is moved to a part of the varicose vein which has not been heated, and FIG. 6B shows a state after the heating portion is moved to the part of the varicose vein which has not been heated.

FIGS. 7A and 7B are schematic sectional views illustrating the first treatment method, wherein FIG. 7A shows a condition where the heated portion has heated the varicose vein, and FIG. 7B shows a state when the heating portion is drawn out of a contracted varicose vein.

FIGS. 8A and 8B are schematic sectional views illustrating a second treatment method, wherein FIG. 8A shows a condition where the heating portion is separated from the part of a varicose vein which has been heated by contracting the heating portion, and FIG. 8B shows a state after the heating portion is moved to a part of the varicose vein which has not been heated.

FIGS. 9A and 9B are schematic sectional views illustrating the second treatment method, wherein FIG. 9A shows a condition where heating portion has moved and expanded to contact the varicose vein, and FIG. 9B shows a condition where the varicose vein has been heated by the moved heating portion.

FIGS. 10A and 10B are schematic sectional views illustrating the second treatment method, wherein FIG. 10A shows a condition where the heating portion is separated from the part of the varicose vein which has been heated by contracting the heating portion, and FIG. 10B shows a state when the heating portion is drawn out of the contracted varicose vein.

FIG. 11 is a plan view showing a medical device according to a second embodiment.

FIG. 12 is a sectional view showing a distal portion of the medical device according to the second embodiment.

FIG. 13 is a plan view showing a condition where a heating portion of the medical device according to the second embodiment has been expanded.

FIG. 14 is a sectional view showing a modification of the medical device according to the first embodiment.

FIG. 15 is a plan view showing a modification of the medical device according to the second embodiment.

FIG. 16 is a development of a heating portion in the modification of the medical device according to the second embodiment.

### DETAILED DESCRIPTION

A medical device and method for heating a body lumen according to the described aspects of the present disclosure will be described in detail below, with reference to several embodiments illustrated in the attached drawings. The embodiments represent examples of the inventive medical device and method for heating a body lumen disclosed here. Note that the dimensional ratios in the drawings may be exaggerated for convenience of explanation and may therefore be different from the actual ratios.

A medical device 10 according to a first embodiment disclosed here is used for therapy (i.e., treatment) of a vein, particularly for a therapy in which a varicose vein is occluded or contracted (i.e., a therapy to occlude or contract the varicose vein). Varicose veins occur mainly in lower limb veins, particularly in great saphenous veins and small saphenous veins, which are superficial veins. Varicose veins can also occur in pelvic, ovarian and spermatic cord veins. The medical device 10 heats a varicose vein from an inner wall surface to harden the varicose vein while occluding or contracting it (i.e., causing the inner diameter of the varicose vein to decrease). The occluded or contracted varicose vein thus inhibits backflow of blood. Note that herein the side toward which the device is inserted into a vein will be referred to as the “distal end” or “distal side” and the side of an operator's proximal operation will be referred to as the “proximal end” or “proximal side.”

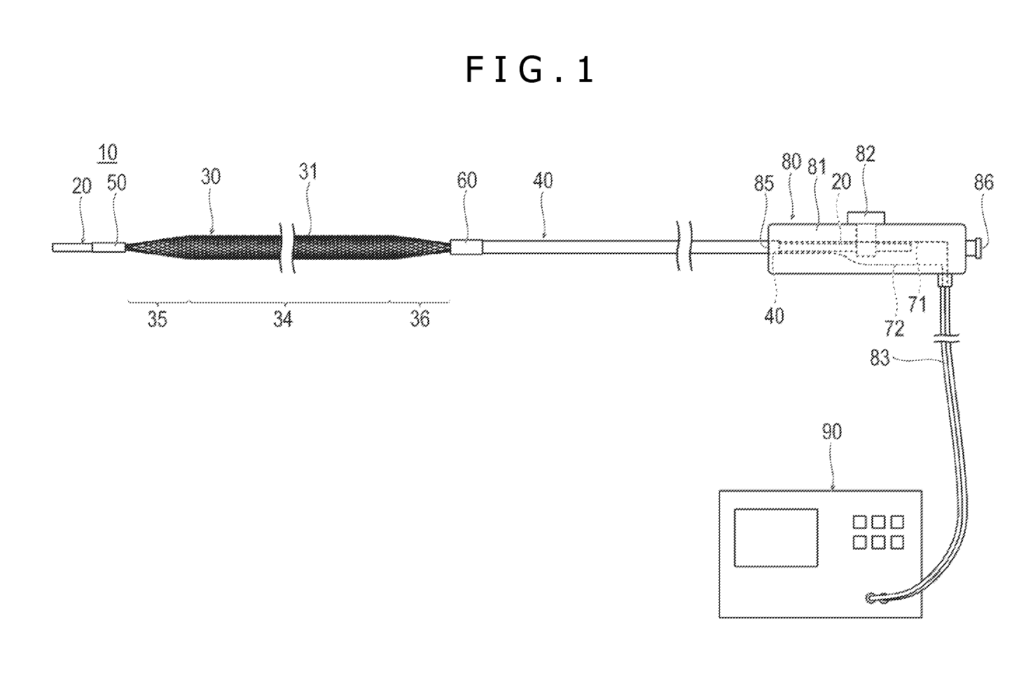

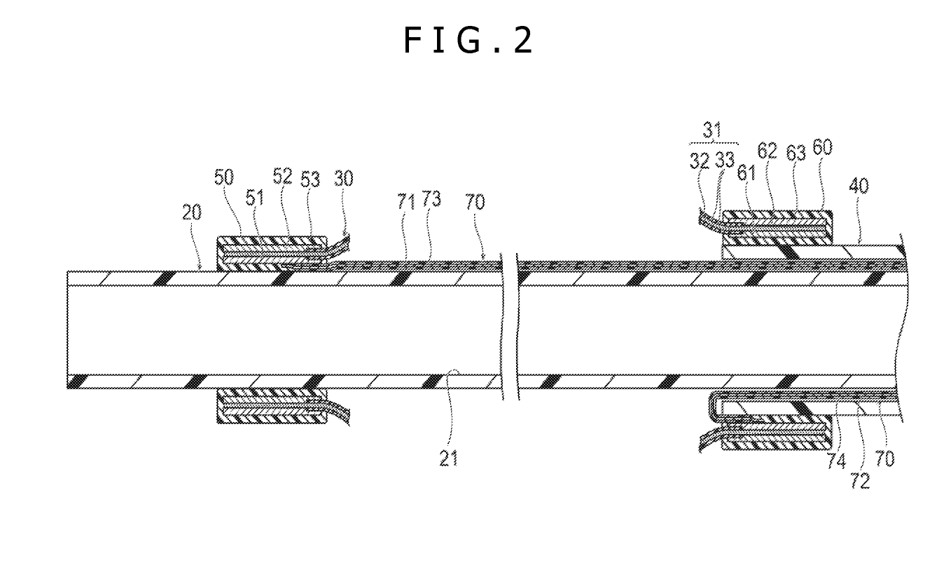

The medical device 10, as shown in FIGS. 1 and 2, includes a pipe shaped inner tube 20 (i.e., an inside shaft portion), a pipe shaped outer sheath 40 accommodating the inner tube 20, a heating portion 30 provided at a distal portion of the medical device 10, leads 70 for supplying a current to the heating portion 30, and an operating portion 80 for operating the heating portion 30.

The inner tube 20 protrudes distally beyond the outer sheath 40. with the inner tube 20 has a guide wire lumen 21 in the inner tube 20 for insertion of a guide wire (i.e., a guide wire is insertable into the guide wire lumen 21).

The outer sheath 40 is a tubular body accommodating the inner tube 20 within the outer sheath 40 (i.e., the inner tube 20 passes through the interior of the outer sheath), and is slidable in an axial direction relative to the inner tube 20.

The distal portion of the heating portion 30 is secured to an outer peripheral surface of a distal portion of the inner tube 20. The proximal portion of the heating portion 30 is secured to an outer peripheral surface of a distal portion of the outer sheath 40. The heating portion 30 includes a plurality of linear material portions 31 braided in a mesh form, a distal-side connection portion 50 to which distal portions of the linear material portions 31 are connected, and a proximal-side connection portion 60 to which proximal portions of the linear material portions 31 are connected.

The plurality of linear material portions 31 are braided (i.e., the linear material portions 31 overlap or intersect one another) to be formed as a whole into a tubular shape having a plurality of voids. Each of the linear material portions 31 includes a conduction portion 32 formed in a spiral shape and capable of conducting a current (i.e., configured to conduct/transfer an electrical current), and a coating portion 33 covering an outer peripheral surface of the conduction portion 32. The coating portion 33 is an insulating material. The distal end and the proximal end of the conduction portion 32 coincide respectively with the distal end and the proximal end of the linear material portion 31 (in other words, the conduction portion 32 is disposed ranging from the distal end to the proximal end of the linear material portion 31 inside of the coating portion 33).

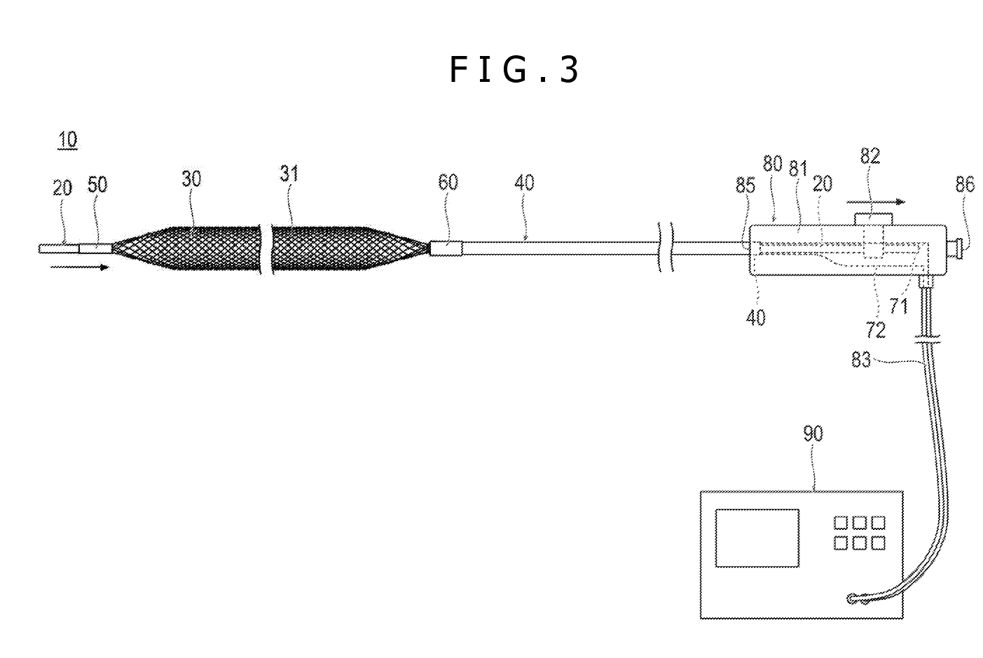

The axially central portion of the heating portion 30, which includes the linear material portions 31, has a tubular portion 34. The tubular portion 34 possesses a substantially cylindrical shape of a substantially constant diameter, so as to ensure that a predetermined range of the heating portion 30 will efficiently contact a body lumen. On the distal side of the tubular portion 34 of the heating portion 30, a first reduced diameter portion 35 is provided which is formed to be tapered (i.e., gradually reduced in diameter) in the distal direction. On the proximal side of the tubular portion 34 of the heating portion 30, a second reduced diameter portion 36 is provided which is formed to be tapered (i.e., gradually reduced in diameter) in the proximal direction. Each of the linear material portions 31 is expandable (i.e., can be expanded while bending to an outer side) radially outward so as to come away from the outer peripheral surface of the inner tube 20, by proximal movement of the inner tube 20 relative to the outer sheath 40 (see FIG. 3). From the expanded state, each linear material portion 31 is contractible (i.e., can be contracted) radially inward so as to come closer to the outer peripheral surface of the inner tube 20, by distal movement of the inner tube 20 relative to the outer sheath 40 (see FIG. 1). The coating portion 33 is made to coat the conduction portion 31 before the linear material portions 31 are braided. Therefore, the coating portions 33 covering the plurality of conduction portions 32 are each configured independently from each other, and are not integrally connected at intersections of the mesh. For this reason, the linear material portions 31 can be flexibly changed in angle at each of the intersections of the mesh, so that the linear material portions 31 can be expanded and contracted easily (i.e., the linear material portions 31 easily expand and contract). A thickness of the coating portion 33 may be able to change along the location of the linear material portions 31. For example, the thickness of the coating portion 33 of the tubular portion 34 possesses a substantially cylindrical shape of a substantially constant diameter, and the thickness of the coating portion 33 of the first reduced diameter portion 35 that is tapered (i.e., gradually reduced in diameter) in the distal direction is thinner than the thickness of the coating portion 33 of the tubular portion 34. On the proximal side of the tubular portion 34 of the heating portion 30, the thickness of the coated portion 33 of the second reduced diameter portion 36 that is tapered (i.e., gradually reduced in diameter) in the proximal direction is thinner than the thickness of the coating portion 33 of the tubular portion 34. The conduction portions 32 can be expanded while bending radially outward (i.e., are expandable radially outward), by axial movement of the inner tube 20 relative to the outer sheath 40. The coating portions 33 covering the outer peripheral surfaces of the conduction portions 32 can bend radially outward in the manner of following up to the conduction portions 32 (i.e., the coating portions 33 expand/bend radially outward along with the conduction portions 32 that the coating portions 33 cover).

The distal-side connection portion 50 includes a pipe shaped (tubular) distal inside connection portion 51, a pipe shaped (tubular) distal outside connection portion 52 coaxially disposed outside of the distal inside connection portion 51 so as to sandwich the linear material portions 31 between the distal outside connection portion 52 and the distal inside connection portion 51, and a distal-side coating portion 53 formed of an insulating material surrounding the outside of the distal inside connection portion 51 and the distal outside connection portion 52. The distal inside connection portion 51 and the distal outside connection portion 52 are conductive. Distal portions of the linear material portions 31 are fixed by being clamped between the pipe shaped distal inside connection portion 51 and the distal outside connection portion 52. The conduction portions 32 are electrically connected to the distal inside connection portion 51 and the distal outside connection portion 52.

The proximal-side connection portion 60 includes a pipe shaped (tubular) proximal inside connection portion 61, a pipe shaped (tubular) proximal outside connection portion 62 coaxially disposed outside of the proximal inside connection portion 61. The outside connection portion 62 thus clamps the linear material portions 31 between itself and the proximal inside connection portion 61. The proximal-side connection portion 60 also includes a proximal-side coating portion 63 formed of an insulating material surrounding the outside of the proximal inside connection portion 61 and the proximal outside connection portion 62. The proximal inside connection portion 61 and the proximal outside connection portion 62 are formed of a conductive material. Proximal portions of the linear material portions 31 are fixed by being clamped between the pipe shaped proximal inside connection portion 61 and the proximal outside connection portion 62. The conduction portions 32 are electrically connected to the proximal inside connection portion 61 and the proximal outside connection portion 62. The conduction portions 32, the distal inside connection portion 51, the distal outside connection portion 52, the proximal inside connection portion 61 and the proximal outside connection portion 62, which are electrically connected as described above, are electrically insulated from the exterior in a reliable manner by being coated with the coating portion 33, the distal-side coating portion 53 and the proximal-side coating portion 63. The coating portions 33, 53, 63 are formed of an insulating material.

The leads 70 include a first lead 71 electrically connected to the distal inside connection portion 51 of the heating portion 30, and a second lead 72 electrically connected to the proximal inside connection portion 61 of the heating portion 30. The first lead 71 is covered with a first coating portion 73 formed of an insulating material, and its distal portion is exposed from the first coating portion 73 and electrically connected to the distal inside connection portion 51 of the heating portion 30. Note that the distal portion of the first lead 71 may be connected to the distal outside connection portion 52 instead of the distal inside connection portion 51 of the heating portion 30, or may be connected directly to the conduction portions 32 on the distal side of the linear material portions 31. The second lead 72 is covered with a second coating portion 74 formed of an insulating material, and its distal portion is exposed from the second coating portion 74 and electrically connected to the proximal inside connection portion 61 of the heating portion 30. Note that the distal portion of the second lead 72 may be connected to the proximal outside connection portion 62 instead of the proximal inside connection portion 61 of the heating portion 30, or may be connected directly to the conduction portions 32 on the proximal side of the linear material portions 31.

The operating portion 80 includes a housing 81, a moving portion 82 movable in an axial direction relative to the housing 81, and a power source cable 83 led out from the housing 81 and connectable to a current supply device 90 which will be described later. A proximal portion of the outer sheath 40 is fixed to the housing of the operating portion 80. The moving portion 82 enters the inside of the housing 81 (i.e., protrudes from outside to inside of the housing 81), and permits the moving portion 82 to move in the axial direction. The housing 81 is has a distal opening 85 as the distal end of the housing 81. The outer sheath 40 is connected to the distal opening 85. The housing 81 has a proximal opening 86 at the proximal end of the housing 81. The proximal opening 86 communicates with the guide wire lumen 21 of the inner tube 20.

The moving portion 82 is partly disposed inside the housing 81, and is partly exposed outside the housing 81. When the exposed part of the moving portion 82 is operated by a finger or fingers (i.e., by a user), the moving portion 82 can be moved in the axial direction relative to the housing 81. A proximal portion of the inner tube 20 penetrating the outer sheath 40 and entering the housing 81 is fixed to the part of the moving portion 82 which is located inside the housing 81 (i.e., a proximal portion of the inner tube 20 is fixed to the moving portion 82 within the housing 81). Therefore, the inner tube 20 can be moved in the axial direction relative to the outer sheath 40 when the moving portion 82 is moved.

Proximal portions of the first lead 71 and the second lead 72 extend into the housing 81 by passing through the gap between the outer sheath 40 and the inner tube 20 (i.e., the leads 71, 72 are located between the outer wall of the inner tube 20 and the inner wall of the outer tube 40). The proximal portions of the first lead 71 and the second lead 72 are electrically connected to the power source cable 83, in the housing 81.

The current supply device 90 connected to the power source cable 83 is a device for supplying a current to be impressed on the heating portion 30. The current supplied may be a direct current or an alternating current, so long as the current can heat the heating portion 30 to a desired temperature.

The materials constituting the inner tube 20 and the outer sheath 40 are preferably materials which have hardness and flexibility. Examples of preferably applicable materials include polyolefins such as polyethylene, polypropylene, etc., polyamides, polyesters such as polyethylene terephthalate, etc., fluoropolymers such as ETFE (ethylene-tetrafluoroethylene copolymer), etc., PEEK (polyether-ether ketone), polyimides, shape memory alloys provided with a shape memory effect or superelasticity by heat treatment, stainless steel, Ta, Ti, Pt, Au, W, and so on. Preferable examples of the shape memory alloys include Ni—Ti alloys, Cu—Al—Ni alloys, and Cu—Zn—Al alloys. Metallic braids or coils may be added to the above-mentioned materials for the purpose of enhancing rigidity.

The inner tube 20 and the outer sheath 40 may be formed to contain a radiopaque material in the material of the inner tube 20 and the outer sheath 40. This enables accurate grasping of positions, and hence an easier procedure, under radioscopy. Preferable examples of the radiopaque material include gold, platinum, platinum-iridium alloy, silver, stainless steel, molybdenum, tungsten, tantalum, palladium, and alloys thereof.

In addition, a marker composed of a radiopaque material may be disposed at a position of either of the inner tube 20 and the outer sheath 40. For example, of the radiopaque marker may be disposed where the inner tube 20 is surrounded by the heating portion 30. The marker may be attached to the inner tube 20 or the outer sheath 40 by a method in which a wire formed of a radiopaque material is wound around the outer surface of the inner tube 20, or a method in which a pipe (tube) formed of a radiopaque material is caulked or adhered onto the outer surface of the inner tube 20.

The material constituting the conduction portions 32 is preferably a material which is electroconductive, is capable of generating heat (i.e., configured to generate heat) through electric resistance, and has both hardness and flexibility. Examples of preferably usable material include shape memory alloys provided with a shape memory effect or superelasticity by heat treatment, stainless steel, Ta, Ti, Pt, Au, W, and so on. Preferable examples of the shape memory alloys include Ni—Ti alloys, Cu—Al—Ni alloys, and Cu—Zn—Al alloys, of which particularly preferred are Ni—Ti alloys. Note that the shape memory alloys such as Ni—Ti alloys are lower in electric resistance than Ni—Cr alloys ordinarily used as material for heat generating elements. Therefore, the cross sectional area and length of the conduction portions 32 are preferably set by taking calorific value (quantity of heat) into account.

The plurality of linear material portions 31 may be partly formed of the above-mentioned radiopaque material.

The materials constituting the housing 81 and the moving portion 82 are not particularly limited. Examples of preferably usable materials include rigid resins such as polycarbonate, polyethylene, polypropylene, etc.

The length of the medical device 10 (the length from a distal-most end of the inner tube 20 to the operating portion 80) is not particularly limited, but is preferably 100 to 1,000 mm, for example. The outside diameter of the outer sheath 40 is not specifically restricted, but is preferably 1.0 to 3.0 mm, for example. The inside diameter of the inner tube 20 is not particularly limited, but is preferably 0.3 to 1.0 mm, for example. The maximum outside diameter of the heating portion 30 in its expanded state is not particularly restricted, but is preferably 3.0 to 20 mm, for example. The length of the heating portion 30 is not specifically limited, but is preferably 3 to 150 mm, for example.

The material or materials constituting the distal inside connection portion 51, the distal outside connection portion 52, the proximal inside connection portion 61 and the proximal outside connection portion 62 are not specifically restricted, so long as the materials are electrically conductive. Examples of preferably applicable materials include iron, copper, aluminum, stainless steel, Ni—Ti alloys, gold, brass, titanium, and so on.

The material or materials constituting the distal-side coating portion 53, the proximal-side coating portion 63, the first coating portion 73 and the second coating portion 74 are not particularly limited, so long as the materials are insulating materials. Examples of preferably usable materials include PTFE (polytetrafluoroethylene), PFA (perfluoroalkoxy fluororesin), FEP (tetrafluoroethylene-hexafluoropropylene copolymer), ETFE, PVDF (polyvinylidene fluoride), PCTFE (polychlorotrifluoroethylene), ECTFE (ethylene-chlorotrifluoroethylene copolymer), silicone resin, and so on.

A first method of using the medical device 10 according to the first embodiment is described below, in relation to an example of occluding a varicose vein occurring in a great saphenous vein or a small saphenous vein of the lower limb.

First, the medical device 10 is primed by replacing air inside the medical device 10 with a physiological salt solution. In this initial state, as shown in FIG. 1, the heating portion 30 is in a contracted state. Next, the power source cable 83 is connected to the power supply device 90.

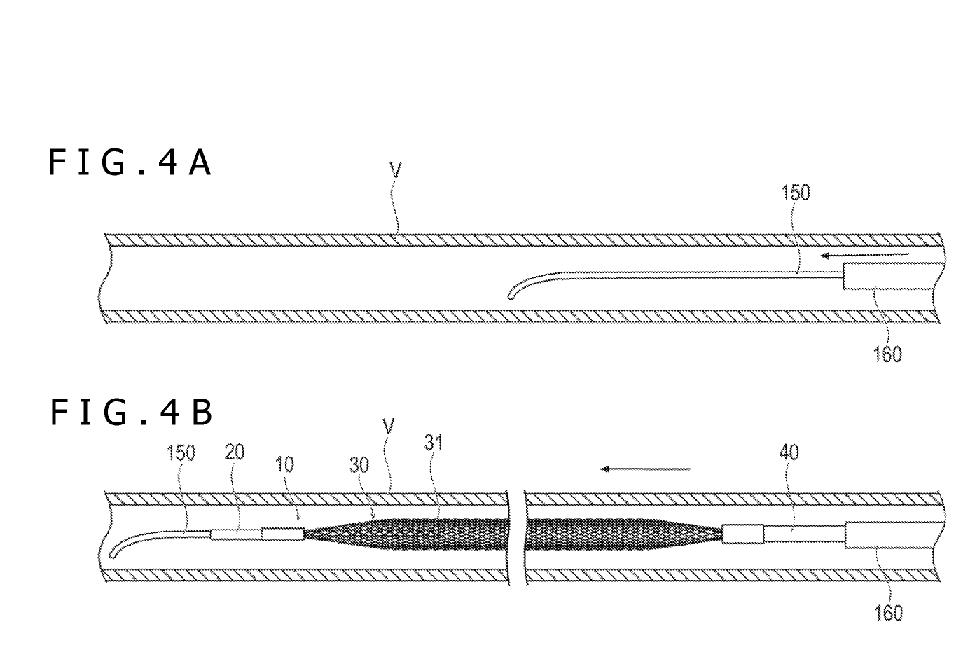

In the case of occluding a great saphenous vein or a small saphenous vein, normally an introducer sheath (not shown) is indwelled into the great saphenous vein or small saphenous vein by way of the knee. The introducer sheath thus provides easy access to the inside of a vein V. A guide wire 150 is inserted into the vein V through the introducer sheath. Next, as shown in FIG. 4A, a guiding catheter 160 is inserted along the guide wire 150 to a desired part. Then, the guide wire 150 is inserted into the guide wire lumen 21 of the medical device 10 prepared in the initial state, and the medical device 10 is inserted into the vein V, starting from its distal portion, in the manner of coming to extend along the guide wire 150. Note that the position at which to set the introducer sheath is not limited to the knee, and the insertion direction may be either an upstream direction or a downstream direction.

Subsequently, as shown in FIG. 4B, the medical device 10 is pushed forward to protrude from the guiding catheter 160, and the heating portion 30 is pushed in to the distal end of the treatment range of the varicose vein (insertion step). The distal end of the treatment range (i.e., a target site in a body lumen) may be, for example, the vicinity of a joining portion of the superficial vein side (the great saphenous vein or small saphenous vein) and a deep vein (for instance, a portion spaced by 10 to 20 mm from the joining portion toward the superficial vein side).

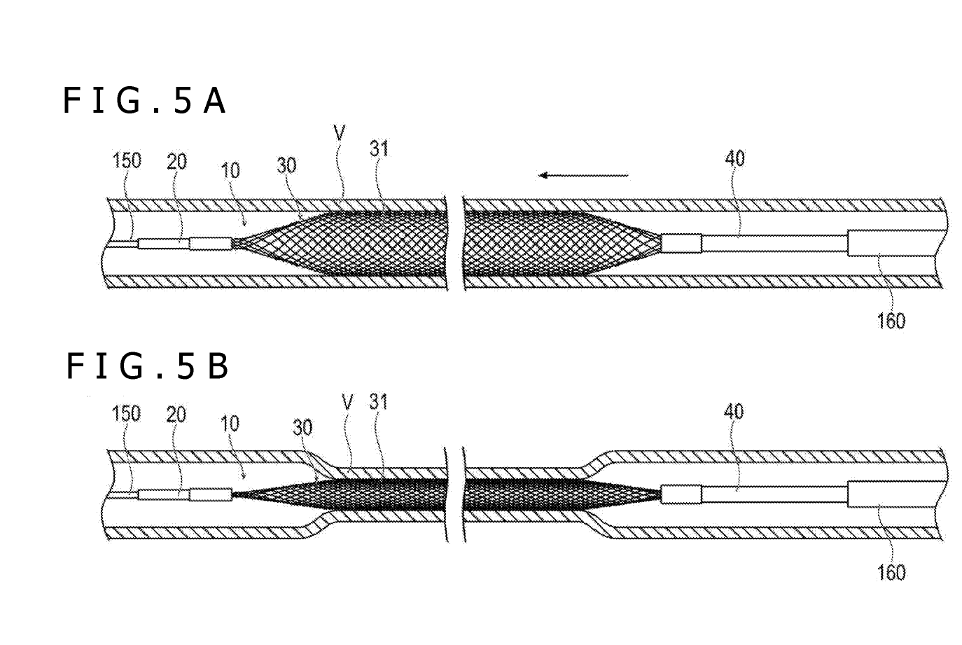

Next, the operating portion 80 is operated to move the moving portion 82 proximally relative to the housing 81. The movement of the operating portion 80 causes the inner tube 20 (to which a distal portion of the heating portion 30 is connected) to move proximally relative to the outer sheath 40 (to which a distal portion of the heating portion 30 is connected as shown in FIGS. 3 and 5A), so that the heating portion 30 is expanded to contact the inner wall surface of the vein V (contact step). Since the heating portion 30 is elastically deformable, the heating portion 30 deforms along the shape of the inner wall surface of the vein V, to make close contact with the vein V. The linear material portions 31 constituting the heating portion 30 may be brought into contact with the inner wall surface of the vein V in the manner of biting into (or engaging) the inner wall surface. This inner wall engagement ensures that at the time of heating the vein V by the heating portion 30, input of heat to blood can be minimized. This helps restrain thrombus formation which might arise from heating of blood, and yet allows the vein V to be heated effectively. The vein V deformed so as to be curved may be expanded to be enlarged in diameter and length in a comparatively rectilinear form.

Subsequently, a current is supplied from the current supply device 90. A current flows through the conduction portions 32 (see FIG. 2) of the heating portion 30 disposed between the first lead 71 and the second lead 72, so that the temperature of the heating portion 30 rises (i.e., increases) owing to the electric resistance of the conduction portions 32. By this process, the inner wall surface of the vein V in contact with the heating portion 30 is heated (first heating step). As described, the heating portion 30 is in contact with the inner wall surface of the vein V through elastic deformation such as to come to lie along the shape of the inner wall surface of the vein V (i.e., the heating portion 30 elastically deforms to take the shape of the inner wall surface of the vein V). Therefore, the vein V can be heated directly, instead of heating the vein V through blood. Accordingly, the vein V can be heated evenly, and the heating temperature can be lowered. Note that for secure contact of the heating portion 30 with the inner wall surface of the vein V while undergoing elastic deformation, the maximum outside diameter of the heating portion 30 when it is expanded by moving the moving portion 82 is preferably greater than the inside diameter of the vein V, on the assumption that the heating portion 30 is expanded without being hindered by the vein V.

The heating temperature of the heating portion 30 is preferably a temperature at which collagen fibers in the vascular media are irreversibly thermally denatured and thermally contracted, for example, 60° C. to 100° C., preferably 70° C. to 80° C. The heating time is, for example, 30 seconds or less. The relationship between the heating temperature and the heating time can be set according to the quantity of heat inputted to the vein V. For example, in order to make constant the quantity of heat determined from the following formula (1) (or a value proportional to the quantity of heat), the heating time can be varied according to the heating temperature (see Table 1).

[Formula (1)]

Quantity of heat inputted=(heating temperature−37° C.)×time (1)

TABLE 1

| Heating temperature (° C.) | Heating time (second) |
|---|---|
| 60 | 24 to 29 |
| 65 | 20 to 24 |
| 70 | 17 to 20 |
| 75 | 14 to 18 |
| 80 | 13 to 15 |
| 85 | 11 to 14 |
| 90 | 10 to 13 |
| 95 | 9 to 12 |
| 100 | 8 to 11 |

Note that since the conduction portions 32 are coated with the insulating coating portions 33, current leakage from the heating portion 30 does not occur. Accordingly, safety for the living body can be enhanced.

When the vein V is heated by the heating portion 30 for a predetermined period of time, the collagen fibers in the vascular media are irreversibly thermally denatured and the vein V contracts (i.e., the inner diameter of the vein V decreases). The heating portion 30 correspondingly contracts, as shown in FIG. 5B. Thus, since the heating portion 30 can be contracted following up to the contraction of the vein V during heating (i.e., the heating portion 30 is elastically deformable to contract in accordance with the shrinked deformation shape of the vein V when the vein V contracts), the contraction of the vein V is not hindered, and the contact of the heating portion 30 with the vein V is maintained constantly. Therefore, the vein V can be effectively heated while minimizing input of heat to blood, and the heating temperature can be made to be about 60° C. to 100° C. Accordingly, enhanced safety can be ensured while mitigating pain, it is unnecessary to apply pressure to the blood vessel from outside of the body, and the vein V can be heated by a simple operation. In addition, since the input of heat to blood can be minimized, thrombus formation which might arise from heating of blood can be restrained.

There is a known method of heating a vein V in which after TLA is applied, a catheter provided with a heating portion is inserted into a vein V and the vein V is heated to about 120° C. According to the present embodiment, on the other hand, since the vein V can be effectively heated while minimizing unevenness of heating by the use of the thermally deformable heating portion 30, the heating temperature can be lowered, whereby safety is enhanced, and the treatment (the heating treatment) can be carried out in a short time without applying TLA.

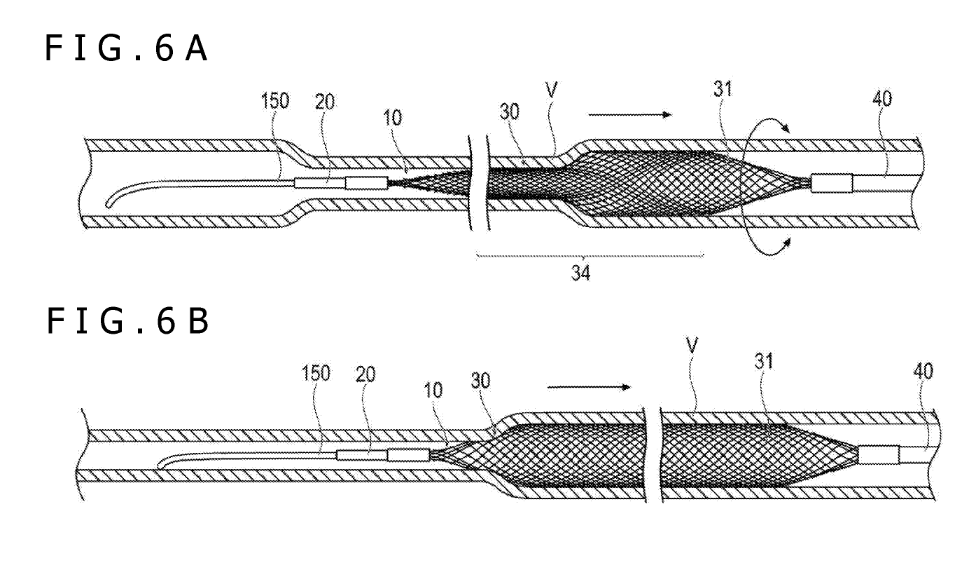

Next, as shown in FIGS. 6A and 6B, the operating portion 80 is operated to move the medical device 10 proximally to move the heating portion 30 to a part of the vein V which has not been heated (and thus not contracted). When the heating portion 30 is moved to the part of the vein V which has not been heated or contracted, the heating portion 30 expands by its own restoring force (i.e., the heating portion 30 self-expands radially outward), and elastically deforms along the shape of the inner wall surface of the vein V, to make contact with the inner wall surface of the vein V (movement step). When the heating portion 30 is moved within the vein V, as shown in FIG. 6A, a distal-side portion of the tubular portion 34 is located within the contracted portion of vein V, whereas a proximal-side portion of the tubular portion 34 is located within the non-contracted portion of vein V. In this case, the interval of the intersections of the linear material portions 31 (i.e., the distance between adjacent intersection points of the linear material portions 31) of the tubular portion 34 in the circumferential direction is the smallest within the contracted portion of vein V, the interval is gradually increased within that portion of vein V at which the inside diameter of vein V gradually increases between the contracted portion of vein V and the non-contracted portion of vein V, and the interval is the greatest within the non-contracted portion of vein V.

At the time of moving the heating portion 30 within the vein V, the operating portion 80 may be operated to rotate the heating portion 30. Such an operation enables the heating portion 30 located within the contracted vein V to be easily moved. The distance the heating portion 30 is moved is preferably not more than the length of that part of the heating portion 30 which contacts the vein V, in order to ensure that a non-heatable part is not generated in the vein V (i.e., to ensure that part of the vein V is not heated/contracted).

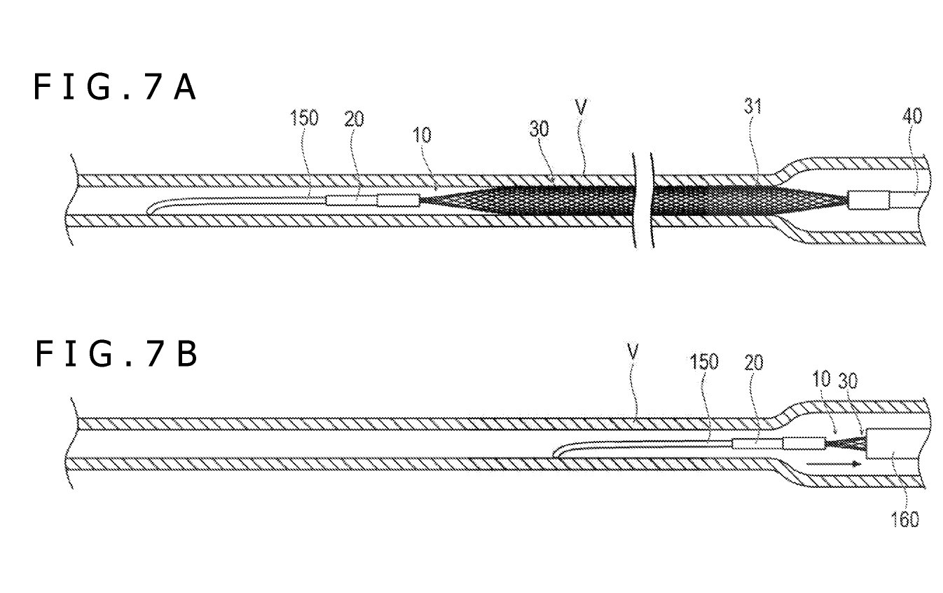

Thereafter, the vein V is heated by the heating portion 30 for a predetermined period of time, whereon the collagen fibers in the vascular media are irreversibly thermally denatured and the vein V is contracted, so that the inside diameter of the vein V is decreased, attended by a decrease in the inside diameter of the heating portion 30 (i.e., the heating portion 30 contracts in accordance with the decreasing of the inner diameter of the vein V) as shown in FIG. 7A (additional heating step).

Thereafter, the movement step of moving the heating portion 30 and the additional heating step of heating the vein V by the heating portion 30 are repeated a predetermined number of times, to occlude or contract a predetermined length of the vein V. Thus, the medical device 10, by repeated movement and heating of the heating portion 30, is capable of occluding or contracting (i.e., configured to occlude or contract) a varicose vein (which is ordinarily as long as 30 to 40 cm) by use of the heating portion 30 (which is, for example, about 7 cm long) shorter than the varicose vein, and is therefore excellent in operability.

After a predetermined length of vein V is occluded or contracted, the moving portion 82 is moved distally relative to the housing 81 to contract the heating portion 30, and the heating portion 30 is drawn out of the contracted vein V (i.e., removed), as shown in FIG. 7B. Thereafter, the medical device 10 is accommodated in the guiding catheter 160, and the medical device 10 is drawn out of (i.e., removed from) the introducer sheath, after which the guiding catheter 160 and the introducer sheath are drawn out of the vein V, to complete the treatment.

A second method of using the medical device 10 according to the first embodiment will now be described.

The second method of using the medical device 10 has the same insertion step of inserting the medical device 10 into the vein V (see FIG. 4B), the contact step of expanding the heating portion 30 to bring the heating portion 30 into contact with the inner wall surface of the vein V (see FIG. 5A), and the heating step of heating the vein V by the heating portion 30 (see FIG. 5B).

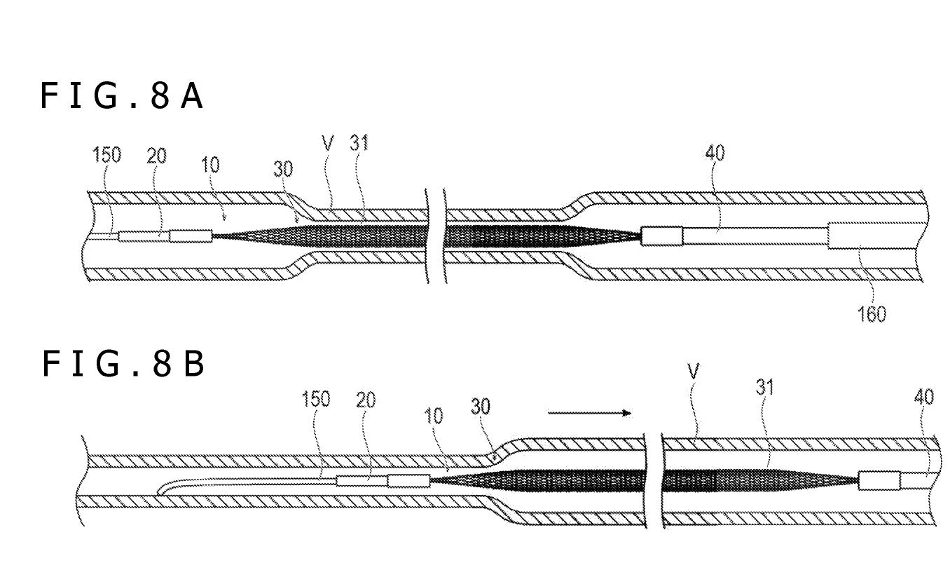

After the vein V is contracted in the heating step, the moving portion 82 is moved distally relative to the housing 81. By this operation, as shown in FIG. 8A, the heating portion 30 is contracted to at least partially come away from the vein V (contraction step) (i.e., the outer diameter of the heating portion 30 is reduced so that the heating portion 30 at least partially does not contact the inner wall of the vein V). Thereafter, the medical device 10 is moved proximally, so that the heating portion 30 is moved to a part of the vein V which has not been heated and contracted, as shown in FIG. 8B (movement step). In the second method, since the heating portion 30 has been contracted, the heating portion 30 can be easily moved without exerting a burden on the vein V (i.e., without applying a force to the inner wall of the vein V). The distance the heating portion 30 is moved is preferably not more than the length of that part of the heating portion 30 which contacts the vein V, in order to ensure that a non-heatable part is not generated in the vein V (i.e., to ensure that part of the vein V is not heated/contracted).

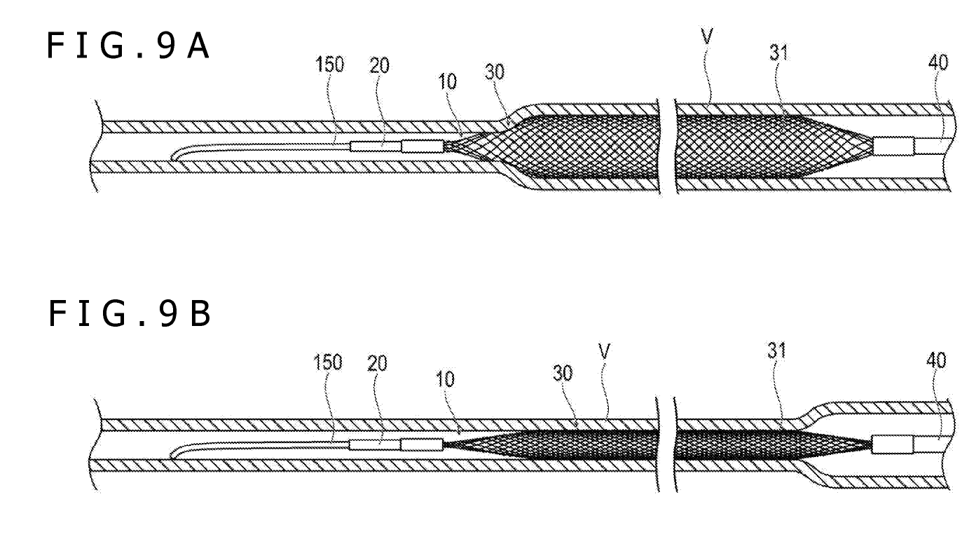

Thereafter, the moving portion 82 is moved proximally relative to the housing 81. By this operation, as shown in FIG. 9A, the inner tube 20 (to which a distal portion of the heating portion 30 is connected) is moved proximally relative to the outer sheath 40 (to which a proximal portion of the heating portion 30 is connected), so that the heating portion 30 is expanded to contact the inner wall surface of the vein V (contact step). Since the heating portion 30 is elastically deformable, the heating portion 30 deforms along the shape of the inner wall surface of the vein V to make close contact with the vein V.

Subsequently, the vein V is heated by the heating portion 30 for a predetermined period of time. Heating the vein V causes the collagen fibers in the vascular media to be irreversibly thermally denatured and the vein V to contract, attended by contraction of the heating portion 30 (i.e., the heating portion 30 contracts in accordance with the decreasing of the inner diameter of the vein V), as depicted in FIG. 9B.

Thereafter, the contraction step of contracting the heating portion 30, the movement step of moving the heating portion 30, the contact step of expanding the heating portion 30 to bring the heating portion 30 into contact with the vein V, and the additional heating step of heating the vein V by the heating portion 30 are repeated a predetermined number of times, to occlude or contract a desired length of vein V. This method allows heating of the vein V without application of TLA.

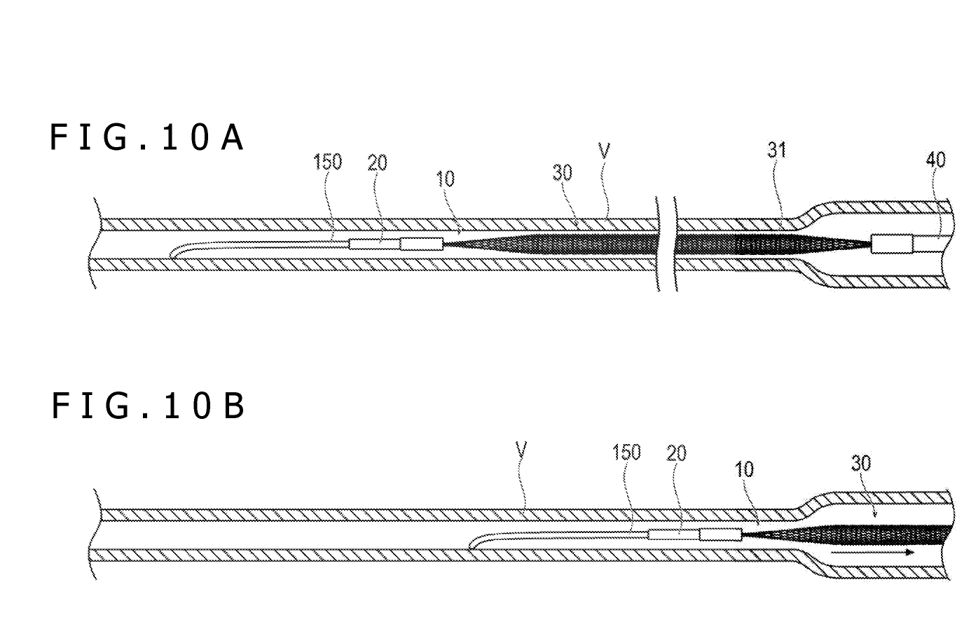

After the desired length of the vein V is occluded or contracted, the moving portion 82 is moved distally relative to the housing 81 to contract the heating portion 30 to at least partially come away from the vein V (i.e., to at least partially not contact the inner wall of the vein V), as shown in FIG. 10A (contraction step). Thereafter, the heating portion 30 is drawn out of the contracted vein V, as shown in FIG. 10B. The medical device 10 is then accommodated in the guiding catheter 160 and drawn out of (i.e., removed from) the introducer sheath, and the guiding catheter 160 and the introducer sheath are drawn out of the vein V, to complete the treatment.

As has been described above, the medical device 10 according to the first embodiment includes: the elongate outer sheath 40; the inner tube 20 (inner shaft portion) extending through the inside of the outer sheath 40 to protrude distally beyond the outer sheath 40; the heating portion 30 disposed between a distal portion of the outer sheath 40 and a distal portion of the inner tube 20, the heating portion 30 including the plurality of linear material portions 31 extending in the axial direction of the inner tube 20, and being formed in a tubular shape as a whole; and the leads 70 electrically connected to the heating portion 30 for passing a current to the heating portion 30. Each of the linear material portions 31 include the conduction portion 32 configured to conduct a current, and the coating portion 33 formed of an insulating material covering the outer peripheral surface of the conduction portion 32. The heating portion 30 can be expanded while bending to the radially outer side (i.e., the heating portion 30 is expandable radially outward) by axial movement of the inner tube 20 relative to the outer sheath 40, and the temperature of the heating portion 30 rises owing to electric resistance when a current flows through the heating portion 30. The medical device 10 configured as above can perform heating by passing a current to the heating portion 30 which can be expanded while bending to the radially outer side. When the temperature of the heating portion 30 increases and the heating portion 30 is deformed along the vein V (i.e., a body lumen) to contact the vein V, the vein V can be directly heated while minimizing input of heat to blood. In addition, since the heating portion 30 can be contracted following up to the contraction of the vein V during heating (i.e., the heating portion 30 contracts radially inward in accordance with the contraction of the vein V), the contraction of the vein V is not obstructed, and an appropriate contact condition can be maintained constantly. Accordingly, the vein V can be effectively heated while minimizing unevenness of heating, so that the heating temperature can be lowered, which enhances safety. It becomes unnecessary to compress the blood vessel from outside the body, and so the vein V can be heated by a simple operation. In addition, input of heat to blood can be minimized, so that thrombus formation can be restrained. Further, since the heating temperature can be lowered, the treatment can be carried out in a short time without applying TLA.

In addition, since the conduction portions 32 are coated by the insulating coating portions 33, leakage of current from the conduction portions 32 to the living body is inhibited. The coating portions 33 thus enhance safety, and the temperature of the heating portion 30 can be efficiently increased.

Since the conduction portion 32 is disposed ranging from the distal end to the proximal end of the inside of the linear material portion 31, the linear material portions 31 themselves, which bend flexibly following up to the vein V (body lumen), can be heated, so that the vein V can be heated effectively.

Further, the heating portion 30 can be elastically deformed by being reduced in outer diameter when pressed from the radially outer side when the relative movement of the inner tube 20 (inside shaft portion) and the outer sheath 40 in the axial direction is restricted (i.e., the inner tube 20 movement is fixed relative to the outer sheath 40). Therefore, the vein V (i.e., a body lumen) can be heated with the heating portion 30 in contact with the vein V, and the heating portion 30 can deform in accordance with the reduction in inside diameter of the vein V. Accordingly, the vein V being deformed can be effectively heated, while appropriately maintaining the contact of the heating portion 30 with the vein V.

In addition, different parts of the heating portion 30 along the axial direction can be deformed into different shapes when pressed from the radially outer side when the relative movement of the inner tube 20 (inside shaft portion) and the outer sheath 40 in the axial direction is restricted. Therefore, when the heating portion 30 has deformed in accordance with the reduction in the inside diameter of the vein V (body lumen) and the heating portion 30 is moved in the axial direction of the vein V, the heating portion 30 can be expanded under its own restoring force (i.e., self-expands) while moving the heating portion 30 to that part of the vein V which has not been heated (i.e., a part of the vein V with a larger inner diameter than the part of the vein V that has been heated). Accordingly, the heating portion 30 can be deformed into a shape following up to the vein V whose inside diameter varies along the axial direction, and the vein V can be heated continuously and with minimized unevenness.

Besides, where the conduction portions 32 are formed of a shape memory alloy, the heating portion 30 can be elastically deformed greatly, so that the heating portion 30 can be elastically deformed in the manner of following up to the vein V which is contracted by heating (i.e., if the conduction portions 32 are a shape memory alloy, the heating portion 30 is more easily elastically deformable to take the shape of the inner wall of the vein V).

Further, the linear material portions 31 are braided and formed in a tubular shape as a whole (i.e., the linear material portions 31 overlap or intersect one another and collectively form a tubular shape). The interval of the intersections of the linear material portions 31 in the circumferential direction decreases as the heating portion 30 is contracted radially (i.e., the distance between adjacent intersection points of the linear material portions 31 decreases as the heating portion 30 is contracted radially inwards). Therefore, the outside diameter of the heating portion 30 can be changed arbitrarily, while deforming the mesh shape of the braided linear material portions 31. Accordingly, when the heating portion 30 is moved within the vein V, the heating portion 30 can constantly keep good contact with the inner wall surface of the vein V whose inside diameter varies, in the manner of following up to the inner wall surface, while the outside diameter of the heating portion 30 is being gradually changed by deformation of the mesh shape of the braided linear material portions 31.

As described above, the leads 70 include the first lead 71 connected to a distal portion of the heating portion 30, and the second lead 72 connected to a proximal portion of the heating portion 30. Therefore, the heating portion 30 can be heated by applying a current between the distal portion and the proximal portion of the heating portion 30, while permitting deformation of the heating portion 30.

In addition, the present disclosure provides a first treatment method for treatment (therapy) of a vein. The first treatment method includes (i) an insertion step of inserting into a body lumen a medical device including a heating portion at a distal portion of the medical device, the heating portion being expandable to a radially outer side through elastic deformation and being configured to generate heat when a current is passed therethrough, (ii) a contact step of expanding the heating portion into a shape along a wall of the body lumen to bring the heating portion into contact with the body lumen wall, (iii) a first heating step of heating the body lumen by the heating portion and deforming the heating portion according to the shape of the body lumen being contracted, (iv) a movement step of moving the heating portion in the axial direction within the body lumen and deforming the heating portion under its own restoring force in the manner of following up to the shape of the body lumen, and (v) an additional heating step of heating the body lumen by the heating portion and deforming the heating portion according to the shape of the body lumen being contracted. The movement step and the additional heating step are repeated at least once. According to the treatment method, the heating portion can be contracted following up to the contraction of the body lumen during heating (i.e., the heating portion contracts in accordance with contraction of the body lumen during heating), so that the contraction of the body lumen is not obstructed, and an appropriate contact condition of the heating portion with respect to the body lumen can be constantly maintained. Therefore, the body lumen can be effectively heated while minimizing unevenness of heating, so that the heating temperature can be lowered and safety can be enhanced. In addition, it is unnecessary to apply a pressure to the body lumen from outside the body, so that the body lumen can be heated by a simple operation. Further, since the movement of the heating portion permits the heating portion to deform under its own restoring force in the manner of automatically following up to the shape of the body lumen, the body lumen can be effectively heated while maintaining the heating portion in contact with the body lumen without unevenness even when the heating portion is moved.

In the heating step, the heating may be carried out without applying anesthesia (TLA, etc.).

In the movement step, the heating portion may be moved while being rotated. This enables the heating portion to be easily moved within the contracted body lumen.

In the contact step, the linear material portions constituting the heating portion may be caused to contact the body lumen wall in the manner of biting into the body lumen wall. This ensures that the quantity of heat inputted to the body fluid (blood) from the heating portion can be minimized, so that the body lumen can be heated effectively. In addition, the heating portion can be expanded to contact the vein to enlarge the vein in diameter and length. By this operation, bending of the vein is canceled, and the vein inner wall can be evenly heated in a rectilinear form.

In addition, the present disclosure provides a second treatment method for treatment (therapy) of a vein. The second treatment method includes (i) an insertion step of inserting into a body lumen a medical device provided at a distal portion thereof with a heating portion which can be expanded while bending to a radially outer side through elastic deformation and which can generate heat when a current is passed therethrough, (ii) a contact step of expanding the heating portion into a shape along a wall of the body lumen to bring the heating portion into contact with the body lumen wall, (iii) a first heating step of heating the body lumen by the heating portion and deforming the heating portion according to the shape of the body lumen being contracted, (iv) a contraction step of contracting the heating portion within the body lumen, (v) a movement step of moving the heating portion in the axial direction, and (vi) an additional heating step of heating the body lumen by the heating portion and deforming the heating portion according to the shape of the body lumen being contracted. The contraction step, the movement step and the additional heating step are repeated at least once. According to the treatment method, the heating portion can be contracted following up to the contraction of the body lumen during heating, so that the contraction of the body lumen is not obstructed, and the contact of the heating portion with the body lumen is constantly maintained while minimizing unevenness. Therefore, the body lumen can be effectively heated, so that the heating temperature can be lowered and safety can be enhanced. In addition, it is unnecessary to compress the body lumen from outside the body, so that the body lumen can be heated by a simple operation. Further, since the heating portion is moved after the heating portion is contracted, the heating portion can be easily moved after the body lumen has contracted to reduce the burden on the body.

In the contact step, the linear material portions constituting the heating portion may be caused to contact the body lumen wall in the manner of biting into the body lumen wall. This ensures that the quantity of heat inputted to the body fluid (blood) from the heating portion can be minimized, so that the body lumen can be heated effectively.

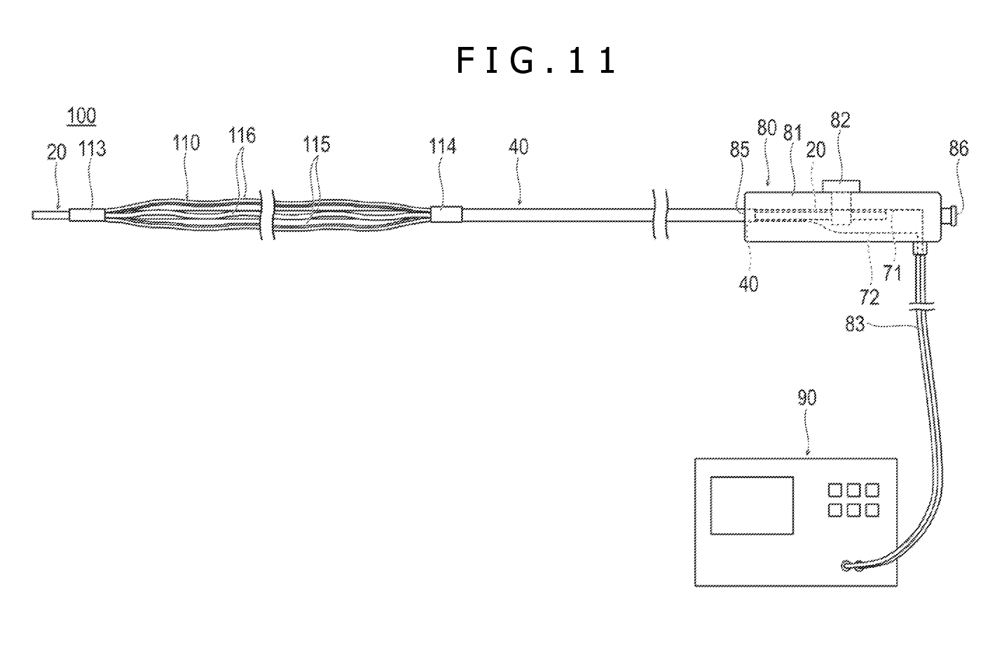

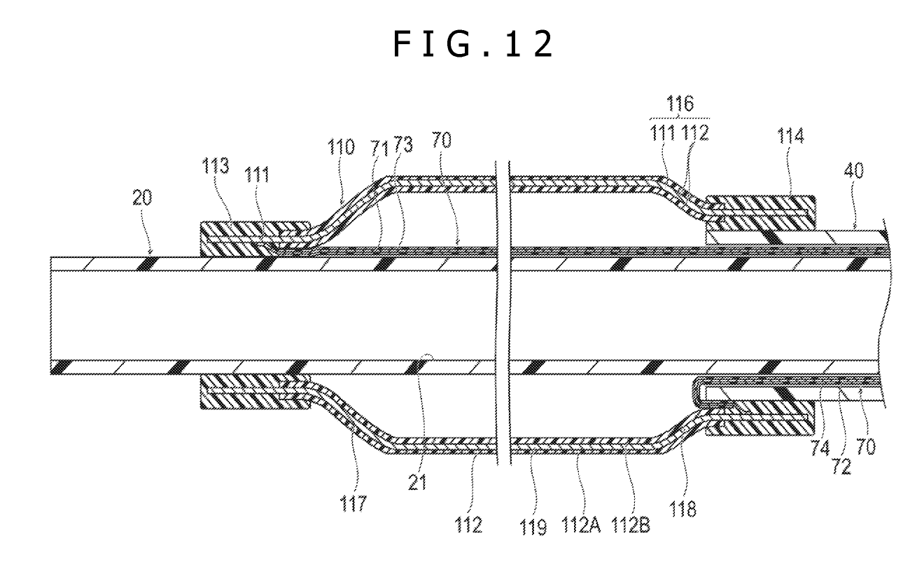

A medical device 100 according to a second embodiment, as shown in FIGS. 11 and 12, differs from the first embodiment only in the configuration of a heating portion 110. Features associated with the second embodiment that are similar to those in the first embodiment described above are identified by common reference numbers and description of those features is omitted.

The heating portion 110 of the medical device 100 according to the second embodiment has a tube body formed with a plurality of slits 115 extending in the axial direction. The slits 115 are arranged evenly in the circumferential direction to form a plurality of linear (filamentous) material portions 116 between the slits 115. The heating portion 110 includes the plurality (e.g., six) of slits 115 extending in the axial direction while curving wavily (i.e., each of the slits 115 curves up and down relative to the medical device 10 along its axial length), the plurality (e.g., six) of linear material portions 116 formed between the slits 115, a distal-side coating portion 113 formed of an insulating material to coat distal portions of the linear material portions 116, and a proximal-side coating portion 114 formed of an insulating material to coat proximal portions of the linear material portions 116. The slits 115 can be easily formed, for example, by subjecting the tube body to laser beam processing. Note that the number of the slits 115 and the linear material portions 116 is not particularly limited.

Each of the linear material portions 116 includes a conduction portion 111 extending in the axial direction and a coating portion 112 formed of an insulating material to coat the conduction portion 111.

The heating portion 110 has its distal portion secured to an outer peripheral surface of a distal portion of the inner tube 20, and has its proximal portion secured to an outer peripheral surface of a distal portion of the outer sheath 40.

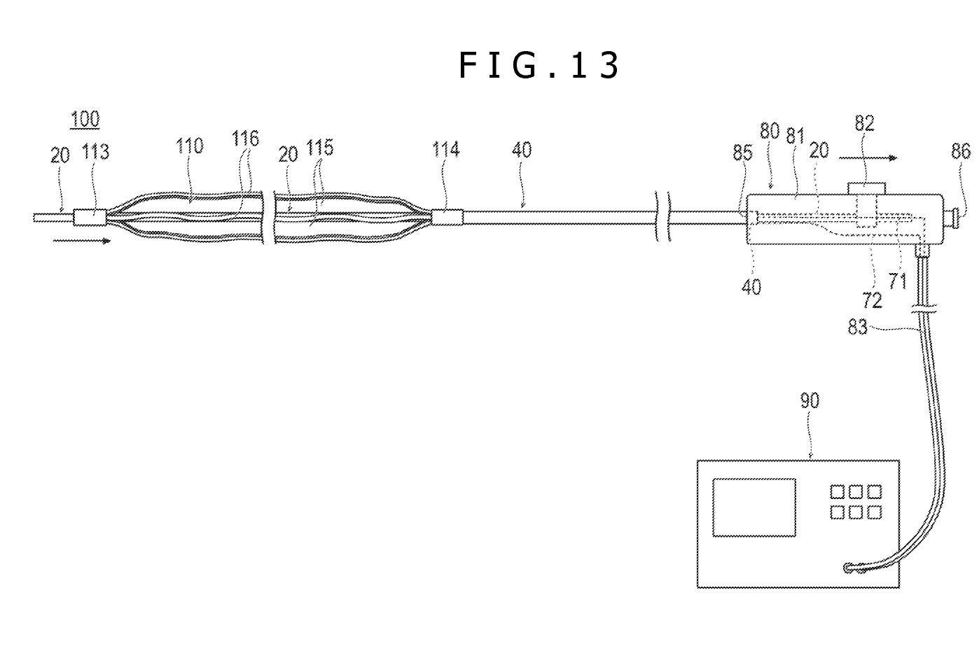

Each linear material portion 116 can be expanded while bending to the outside in the manner of coming away from the outer peripheral surface of the inner tube 20 (i.e., each linear material portion 116 is expandable radially outward), by proximal movement of the inner tube 20 relative to the outer sheath 40 (see FIG. 13). From the expanded state, the linear material portion 116 can be contracted to move closer to the outer peripheral surface of the inner tube 20 by the inner tube 20 moving distally relative to the outer sheath 40 (see FIG. 11). Since each of the linear material portions 116 is curved wavily, the range of contact of the heating portion 110 with an inner wall surface of a vein V when the heating portion 110 is expanded can be dispersed instead of being concentrated into a part, so that the vein V can be occluded or contracted through heating with minimized unevenness.

The distal portion of the conduction portion 111 is exposed from the coating portion 112 and electrically connected to a distal portion of the first lead 71, and the proximal portion of the conduction portion 111 is exposed from the coating portion 112 and electrically connected to a distal portion of the second lead 72. The conduction portion 111, in the expanded state, has a distal conduction portion 117 that is gradually reduced in diameter along the distal direction (i.e., the distal conduction portion 117 is tapered in the distal direction). The conduction portion 111 has a proximal conduction portion 118 that is gradually reduced in diameter along the proximal direction when the conduction portion 111 is expanded. The conduction portion 111 has a central conduction portion 119 between the distal conduction portion 117 and the proximal conduction portion 118. The distal conduction portion 117 and the proximal conduction portion 118 are formed to be thicker (i.e., possess walls with a thicker diameter) than the central conduction portion 119. Therefore, the sectional area in a section orthogonal to the axial direction of the central conduction portion 119 is smaller than the sectional areas in the section orthogonal to the axial direction of the distal conduction portion 117 and the proximal conduction portion 118. Consequently, the electric resistance of the central conduction portion 119 is greater than the electric resistances of the distal conduction portion 117 and the proximal conduction portion 118. In addition, the thickness of the coating portion 112 covering the distal conduction portion 117 and the proximal conduction portion 118 is greater than the thickness of the coating portion 112 covering the central conduction portion 119. The thickness of a coating portion 112A covering the outer peripheral surface side of the central conduction portion 119 is smaller than the thickness of a coating portion 1128 covering the inner peripheral surface side of the central conduction portion 119. Therefore, of the coating portion 112, only the coating portion 112A coming into contact with a wall of a body lumen is formed to be thinner that the other coating portions.

The distal-side coating portion 113 is formed of an insulating material coating a distal portion of the conduction portion 111 together with the first lead 71. The proximal-side coating portion 114 is formed of an insulating material coating a proximal portion of the conduction portion 111 together with the second lead 72.

The conduction portions 111 of the heating portion 110 are electrically insulated from the exterior in a reliable manner, since they are coated with the coating portion 112, the distal-side coating portion 113 and the proximal-side coating portion 114 which are formed of an insulating material.

In the medical device 100 according to the second embodiment, like in the first embodiment, it is possible to elastically expand the heating portion 110 and to bring the heating portion 110 into contact with the inner wall surface of the vein V along the shape of the inner wall surface by moving the inner tube 20 in the axial direction relative to the outer sheath 40. Therefore, the vein V can also be occluded or contracted using the medical device 100 in the second embodiment by utilizing the first treatment method or the second treatment method described in the first embodiment above.

In addition, the distal conduction portion 117 at a distal portion of the conduction portion 111 is gradually reduced in diameter along the distal direction, the proximal conduction portion 118 at a proximal portion of the conduction portion 111 is gradually reduced in diameter along the proximal direction, and the central conduction portion 119 of the conduction portion 111 is between the distal conduction portion 117 and the proximal conduction portion 118. The thickness of the coating portion 112 covering at least one of the distal conduction portion 117 and the proximal conduction portion 118 is greater than the thickness of the coating portion 112 covering the central conduction portion 119. Therefore, while heat is effectively transmitted to the vein V from the central conduction portion 119 in contact with the vein V, the quantity of heat transmitted to the exterior through the thick coating portion 112 from the distal conduction portion 117 and the proximal conduction portion 118, which are liable to contact blood without contacting the vein V, can be reduced, and, accordingly, thrombus formation which might arise from heating of blood can be restrained.

When the sectional area (i.e., thickness) of at least one of the distal conduction portion 117 and the proximal conduction portion 118 is greater than the sectional area (i.e., thickness) of the central conduction portion 119, the electric resistance of the central conduction portion 119 is higher than the electric resistance of the distal conduction portion 117 and/or the proximal conduction portion 118. This ensures that heat is generated effectively at the central conduction portion 119 located in the range of contact of the conduction portion 111 with the vein V, and that, at the distal conduction portion 117 and the proximal conduction portion 118 located in the range where the conduction portion 111 is liable to contact blood without contacting the vein V, the quantity of heat generated is less. Therefore, thrombus formation can be restrained.

Further, since the thickness of the coating portion 112A covering the outer peripheral surface side of the central conduction portion 119 is smaller than the thickness of the coating portion 1128 covering the inner peripheral surface side of the central conduction portion 119, it is ensured that the quantity of heat transmitted to the exterior is reduced at the coating portion 1128 that is liable to contact blood without contacting the vein V. Thrombus formation can thus be restrained, and the vein V can be heated efficiently at the coating portion 112A that contacts the vein V.

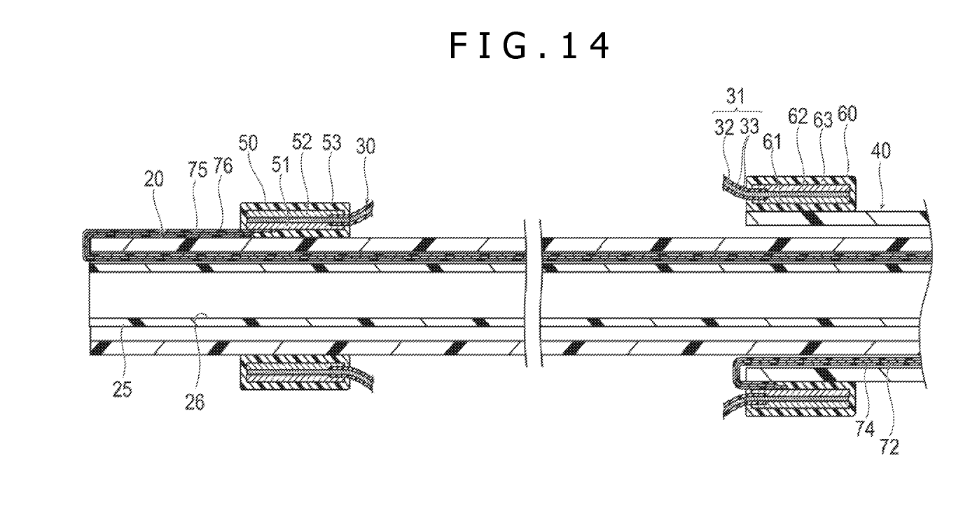

Note that the present disclosure is not to be limited to the aforementioned embodiments, and various modifications can be made by one skilled in the art without departing from the scope of technical thought of the present disclosure. For instance, a first lead 75 covered with a first coating portion 76 may extend through the inside of the inner tube 20 to the operating portion 80, as in a modification of the first embodiment depicted in FIG. 14, instead of extending between the inner tube 20 and the outer sheath 40. In this modification, it is preferable that another second inner tube 25 be disposed inside the inner tube 20 and a guide wire lumen 26 be formed inside the second inner tube 25. Note that parts having functions equivalent or similar to those in the first embodiment are denoted by the same reference symbols as used above, and description of those portions is omitted.

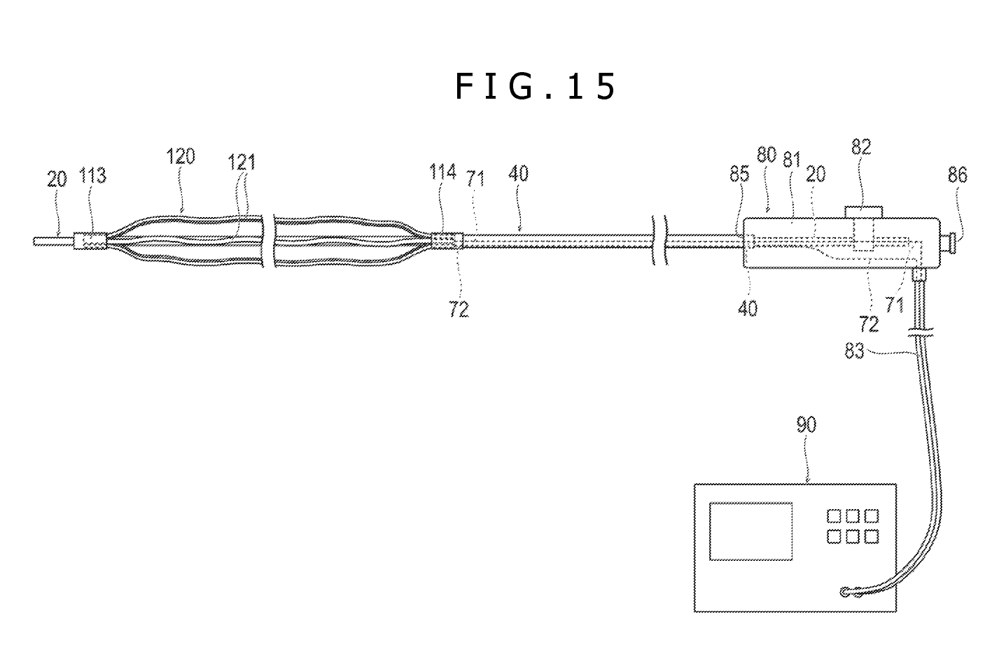

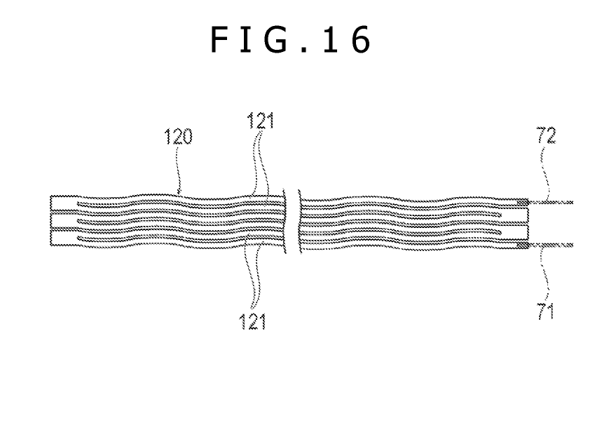

In addition, the configuration of the heating portion is not specifically restricted, so long as the heating portion generates heat when supplied with a current and can be expanded radially outward by movement of the inner tube 20 relative to the outer sheath 40. For example, in a first modification of the second embodiment illustrated in FIGS. 15 and 16, linear material portions 121 constituting a heating portion 120 may be connected to be a single conductor which is alternately folded back at both ends in the axial direction of the heating portion 120. Such a configuration enables both the first lead 71 and the second lead 72 to be disposed juxtaposedly (i.e., side by side) on the proximal side of the heating portion 120, so that the configuration is simplified. Note that the parts of the heating portion 120 for connection with the first lead 71 and the second lead 72 may be set at the distal side of the heating portion 120, instead of the proximal side, so as to allow the first lead 71 and the second lead 72 to be disposed juxtaposedly (i.e., side by side) on the distal side.

In the medical devices 10 and 100 according to the aforementioned first and second embodiments, the vein V is heated while the heating portion 30 or 110 is pulled proximally. However, the vein V may be heated while the heating portion 30 or 110 is pushed in distally.

The operating portion 80 in the medical devices 10 and 100 according to the first and second embodiments has a configuration wherein the inner tube 20 is moved relative to the outer sheath 40 by moving the moving portion 82 to which the inner tube 20 is connected. However, the structure for moving the inner tube 20 is not limited to this structure. For example, a structure may be adopted wherein the housing is provided with a dial capable of being rotated with a finger or fingers to move the inner tube 20 relative to the outer sheath 40, such that when the dial is rotated, the inner tube 20 is moved in the axial direction by a rotating force of the dial.

Instead of the pipe shaped inner tube 20, a solid member (inside shaft portion) which is not formed therein with a lumen may be used.

Further, the body lumen for which the medical devices 10 and 100 according to the first and second embodiments are to be used is not limited to a vein, but may be an artery, or other vessel than blood vessel, or ureter or the like.

The detailed description above describes a medical device and a method for heating a body lumen. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an elongate outer sheath possessing a distal end and an interior;
an inner shaft portion extending through the interior of the outer sheath in an axial direction to protrude distally beyond the outer sheath, the inner shaft portion possessing a distal portion;
a tubular heating portion located between the distal end of the outer sheath and the distal portion of the inner shaft portion, the heating portion comprising a plurality of linear material portions extending in the axial direction of the inner shaft portion, each of the linear material portions possessing an outer peripheral surface;
leads electrically connected to the heating portion to pass current to the heating portion;
each of the linear material portions including a conduction portion configured to conduct current, and a coating portion covering the outer peripheral surface of each of the linear material portions, the coating portion being an insulating material; and
the heating portion being expandable radially outward by moving the inner shaft portion in the axial direction relative to the outer sheath, and a temperature of the heating portion increasing as a result of current flowing through the leads and through the heating portion.

2. The medical device according to claim 1, wherein
the linear portion possesses a distal end and a proximal end at opposite axial ends of the linear portion, and
the conduction portion extending from the distal end to the proximal end of the linear material portion.

3. The medical device according to claim 2, wherein the heating portion is elastically deformable so that an outer diameter of the heating portion is reduced when the heating portion is pressed from a radially outer side of the heating portion and when relative movement of the inner shaft portion and the outer sheath in the axial direction is restricted.

4. The medical device according to claim 3, wherein different portions of the heating portion along the axial direction are deformable into different shapes by being pressed from the radially outer side when relative movement of the inner shaft portion and the outer sheath in the axial direction is restricted.

5. The medical device according to claim 1, wherein
the linear material portions are braided to collectively form a tubular shape,
the linear material portions overlap one another at intersection points to form the tubular shape, and
a distance between adjacent intersection points of the linear material portions in a circumferential direction is reduced as the heating portion is contracted radially inward.

6. The medical device according to claim 1, wherein the leads include a first lead connected to a distal portion of the heating portion, and a second lead connected to a proximal portion of the heating portion.

7. A medical device comprising:
an outer sheath comprising a distal end and an interior;
an inner shaft portion extending through the inner portion of the outer sheath in an axial direction and protruding distally beyond the outer sheath, the inner shaft portion being slidably movable relative to the outer sheath, the inner shaft portion comprising a distal portion located distal to the distal end of the outer sheath in the axial direction;
a heating portion connected to the distal end of the outer sheath and the distal portion of the inner shaft portion, the heating portion comprising a braided mesh;
the braided mesh being comprised of a plurality of linear material portions extending in the axial direction of the inner shaft portion, each of the linear material portions possessing a spiral shape;
each of the linear material portions including a conduction portion configured to conduct current to increase a temperature of the heating portion, the conduction portion of each linear material portion possessing an outer peripheral surface, and a coating portion covering the outer peripheral surface of the conduction portion of each linear material portion, the coating portion being an insulating material; and
the braided mesh of the heating portion being expandable radially outward when the inner shaft portion moves in the axial direction relative to the outer sheath, and the conduction portion conducting current to the heating portion to increase the temperature of the heating portion.

8. The medical device according to claim 7, wherein
the heating portion is elastically deformable;
the heating portion possess an outer diameter and a radially outer side; and
the outer diameter of the heating portion decreases when the heating portion is pressed from the radially outer side of the heating portion and the inner shaft portion is fixed relative to the outer sheath in the axial direction.

9. The medical device according to claim 7, wherein
the linear material portions overlap one another at intersection points to form the braided mesh, and
a distance between adjacent intersection points of the linear material portions in a circumferential direction is reduced as the heating portion is contracted radially inward.

10. The medical device according to claim 7, further comprising leads connected to the conduction portion, the leads configured to conduct the current.

11. The medical device according to claim 10, wherein the leads include a first lead connected to a distal portion of the heating portion, and a second lead connected to a proximal portion of the heating portion.

12. The medical device according to claim 11, wherein
the inner shaft portion possesses an outer wall extending in the axial direction;
the outer sheath possesses an inner wall extending in the axial direction; and
the leads extend in the axial direction between the outer wall of the inner shaft portion and the inner wall of the outer sheath.

13. A method comprising:
inserting a medical device into a living body, the medical device comprising a heating portion possessing an outer peripheral surface;
moving the medical device to a body lumen within the living body, the body lumen possessing an inner wall and an inner diameter;
outwardly expanding the heating portion of the medical device to bring the outer peripheral surface of the heating portion into contact with the inner wall of the body lumen;
heating the inner wall of the body lumen using the heating portion of the medical device while the heating portion is contacting the inner wall of the body lumen; and
contracting the inner wall of the body lumen through the heating of the inner wall of the body lumen with the heating portion so that the inner diameter of the body lumen decreases.

14. The method according to claim 13, wherein the medical device further comprises an outer sheath and an inner shaft portion extending through an inner portion of the outer sheath in an axial direction, the inner shaft portion being movable in the axial direction relative to the outer sheath; and
the outward expansion of the heating portion into contact with the inner wall of the body lumen comprises moving the inner shaft portion in the axial direction relative to the outer sheath.

15. The method according to claim 14, further comprising
contracting the heating portion to decrease an outer diameter of the heating portion after the inner wall of the body lumen is contracted, the outer diameter of the heating portion being smaller than the inner diameter of the body lumen; and
moving the medical device to a different area of the body lumen.

16. The method according to claim 13, wherein the heating portion possesses an outer diameter, and the outer diameter of the heating portion decreases when the inner wall of the body lumen contracts, the method further comprising:
moving the medical device in the axial direction after the inner wall of the body lumen contracts, the heating portion being in contact with the inner wall of the body lumen while the medical device moves; and
the outer diameter of the heating portion being greater than the inner diameter of the body lumen when the medical device is moved in the axial direction after the inner wall of the body lumen contracts.
